(19) United States
(12) Patent Application Publication
(10) Pub. No.: US 2025/0386777 A1
(43) Pub. Date: Dec. 25, 2025

(54) MYCELIUM-BASED BIOCOMPOSITES

(71) Applicant: Khalifa University of Science and Technology, Abu Dhabi (AE)

(72) Inventors: Blaise L. TARDY, Abu Dhabi (AE); Zainuddin ZIYAN, Abu Dhabi (AE); Mohamed Hamid SALIM, Abu Dhabi (AE); AlBlooshi NASER, Abu Dhabi (AE); Ghanim Mabrook Firuz MABROOK, Abu Dhabi (AE); Sarath Haridas KANIYAMPARAMBIL, Abu Dhabi (AE); Srinivas METTU, Abu Dhabi (AE)

(21) Appl. No.: 19/239,243

(22) Filed: Jun. 16, 2025

Related U.S. Application Data
(60) Provisional application No. 63/661,692, filed on Jun. 19, 2024.

(51) Int. Cl. A01G 18/20; A01G 18/10; A01G 18/40; A01G 18/50

(52) U.S. Cl. CPC A01G 18/20; A01G 18/10; A01G 18/40; A01G 18/50

(57) ABSTRACT

A mycelium-based biocomposite comprising mycelium and a substrate including one or more textiles wherein the mycelium is integrated into the substrate and a method for making a mycelium using one or more textiles as a substrate. Growth of mycelial cells into networks throughout the substrate provide the biocomposite with improved morphological and structural properties.

### TECHNICAL FIELD

The subject matter disclosed herein relates to mycelium-based biocomposites and methods of making mycelium-based biocomposites using textiles.

### BACKGROUND

Textile waste management is a growing global problem exacerbated by the continuous growth of the textile industry. A common source of textile waste comes from consumer use, degradation, and disposal of clothing. Common disposal techniques incineration and landfilling can have serious negative effects on the environment and the economy. Moreover, because textile waste often consists of a complex mixture of components, including natural fibers like cotton, synthetic fibers like polyester and nylon, as well as a large variety of blends, it can be extremely difficult to recycle and reuse. Other waste products such as agricultural waste have been repurposed as substrates for mycelium cultivation and formation into useful biocomposites, but this has not been attempted regarding textile waste.

### SUMMARY

According to one aspect, a method for making a mycelium-based biocomposite comprising providing a substrate including one or more textiles, providing mycelium, applying the mushroom spawn to the one or more textiles to form a composite, and allowing the mycelium to grow in the composite to form a mycelium biocomposite.

According to another aspect, a mycelium-based biocomposite comprising a substrate including one or more textiles and mycelium, wherein the mycelium is integrated into the substrate.

### BRIEF DESCRIPTION OF THE DRAWINGS

This written disclosure describes illustrative embodiments that are non-limiting and non-exhaustive. Reference is made to illustrative embodiments that are depicted in the figures, in which:

FIG. 1 illustrates a flowchart showing method 100 for making a mycelium-based biocomposite, according to some embodiments.

FIG. 2A illustrates a light microscopy image of a textile made of cotton.

FIG. 2B illustrates a light microscopy image of biocomposite made with cotton, according to some embodiments.

FIG. 2C illustrates a light microscopy image of biocomposite made with cotton, according to some embodiments.

FIG. 2D illustrates a light microscopy image of a textile made of polyester.

FIG. 2E illustrates a light microscopy image of biocomposite made with polyester, according to some embodiments.

FIG. 2F illustrates a light microscopy image of biocomposite made with polyester, according to some embodiments.

FIG. 3A illustrates a photograph of a heart-shaped biocomposite, according to some embodiments.

FIG. 3B illustrates a photograph of a cuboid-shaped biocomposite, according to some embodiments.

FIG. 4 illustrates a graph showing results of shrinkage analysis for various biocomposites, according to some embodiments.

FIG. 5A illustrates an SEM image of a textile made of cotton.

FIG. 5B illustrates an SEM image of biocomposite made with cotton, according to some embodiments.

FIG. 5C illustrates an SEM image of biocomposite made with cotton, according to some embodiments.

FIG. 5D illustrates an SEM image of biocomposite made with cotton, according to some embodiments.

FIG. 5E illustrates an SEM image of a textile made of polyester.

FIG. 5F illustrates an SEM image of biocomposite made with polyester, according to some embodiments.

FIG. 5G illustrates an SEM image of biocomposite made with polyester, according to some embodiments.

FIG. 5H illustrates an SEM image of biocomposite made with polyester, according to some embodiments.

FIG. 6 illustrates an SEM image of a biocomposite made with lint from several textiles.

FIG. 7A illustrates a load-displacement graph for various biocomposites, according to some embodiments.

FIG. 7B illustrates a load-displacement graph for various biocomposites, according to some embodiments.

FIG. 8A illustrates a graph showing flexural strength for various biocomposites, according to some embodiments.

FIG. 8B illustrates a graph showing ultimate strain for various biocomposites, according to some embodiments.

FIG. 8C illustrates a graph showing toughness for various biocomposites, according to some embodiments.

FIG. 9 illustrates an image array showing results of flammability tests for various textiles and biocomposites, according to some embodiments.

FIG. 10A illustrates a graph showing burn area results from flammability tests for various textiles and biocomposites, according to some embodiments.

FIG. 10B illustrates a graph showing burn area results from flammability tests for various textiles and biocomposites, according to some embodiments.

FIG. 11A illustrates a graph showing combustion rate results from flammability tests for various textiles and biocomposites, according to some embodiments.

FIG. 11B illustrates a graph showing combustion rate results from flammability tests for various textiles and biocomposites, according to some embodiments.

FIG. 12A illustrates a graph showing thermogravimetric results from thermogravimetric analysis for various textiles and biocomposites, according to some embodiments.

FIG. 12B illustrates a graph showing derivative thermogravimetric results from thermogravimetric analysis for various textiles and biocomposites, according to some embodiments.

FIG. 13A illustrates a graph showing results from humidity absorption analysis for various textiles and biocomposites, according to some embodiments.

FIG. 13B illustrates a graph showing results from humidity absorption analysis for various textiles and biocomposites, according to some embodiments.

FIG. 13C illustrates a graph showing results from humidity absorption analysis for various textiles and biocomposites, according to some embodiments.

FIG. 13D illustrates a graph showing results from humidity absorption analysis for various biocomposites, according to some embodiments.

FIG. 14A illustrates a graph showing results from water contact angle analysis for various textiles and biocomposites, according to some embodiments.

FIG. 14B illustrates a graph showing results from water contact angle analysis for various textiles and biocomposites, according to some embodiments.

### DETAILED DESCRIPTION

The present disclosure provides mycelium-based biocomposites incorporating textiles and methods for making mycelium-based biocomposites using textiles as a substrate. Mycelium-based biocomposites comprise a network of mycelial cells within a textile substrate. The mycelial network provides structural support to the biocomposite due to its chitinous composition. The use of textiles as a substrate provides additional beneficial structural and flexural characteristics to biocomposites due to the inherent mechanical characteristics of textiles. Further, this use of textiles allows for additional avenues of reuse of waste materials such as textile waste.

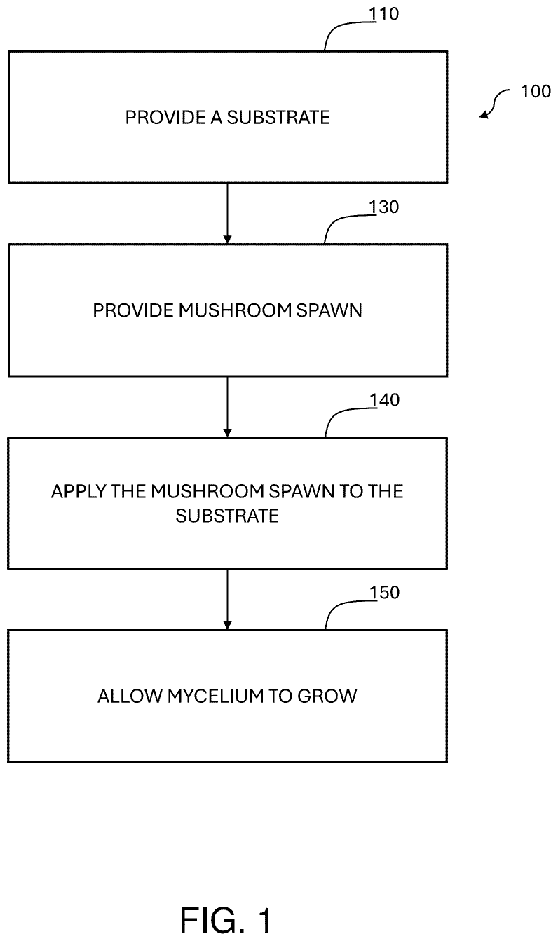

FIG. 1 illustrates a flowchart showing method 100 for making mycelium-based biocomposites, according to some embodiments. Method 100 involves the following steps: providing a substrate 110 including one or more textiles, providing mycelium 120, applying 130 the mycelium to the substrate to form a composite, and allowing the mycelium to grow 140 to form a mycelium biocomposite.

Providing a substrate 110 involves providing a substrate including one or more textiles. Substrates are materials upon which mycelium grows and from which mycelium extracts nutrients. Textiles are materials made from a multiplicity of fibers. Textiles may refer to cloth, fabric, or other related fibrous materials.

Rate and breadth of mycelium growth may be affected by the type of substrate used. In some embodiments, textiles are made from natural fibers, synthetic fibers, or some combination therefrom. In some embodiments, natural fibers include naturally occurring or naturally derived fibers from plants or animals.

In some embodiments, textiles have high nutrient availability for mycelium. In some embodiments, mycelium readily degrade and metabolize the substrate and may undertake a mode of growth characterized by slow expansion with dense aggregation. Many mycelia produce cellulase, specialized enzymes that break down cellulose. As a result, many cellulose-based textiles have high nutrient availability to mycelium that produce cellulase. In some embodiments, the one or more textiles contains one or more cellulose-based fibers selected from cotton, flax, linen, hemp, rayon, lyocell, and modal.

In some embodiments, textiles have low or no nutrient availability for mycelium. In some embodiments, mycelium do not readily degrade and metabolize the substrate and may undertake a mode of growth characterized by fast-growing, elongated branches with loose aggregations. Many non-cellulose-based textiles have low nutrient availability to mycelium that produce cellulase. In some embodiments, the one or more textiles contains one or more non-cellulose-based fibers selected from polyamides, polyolefins, polyesters, polyurethanes, acrylonitrile, and other polymer-based textiles.

In some embodiments, textiles are hygroscopic. Hygroscopic substrates may provide mycelium with beneficial environments in which to grow by absorbing and retaining moisture from the atmosphere.

In some embodiments, textiles are porous. In some embodiments, textiles have an average pore size of at least, equal to, or between any two of 10, 20, 30, 40, 50, 60, 70, 80, 90 and 100 μm. Having a pore size of at least 10 μm allows integration of the mycelium with the substrate via growth through pores.

In some embodiments, textiles include cotton. In some embodiments, textiles include at least, equal to, or between any two of 50%, 60%, 70%, 80%, 90%, or 100% cotton. In some embodiments, textiles include polyester. In some embodiments, textiles include at least, equal to, or between any two of 50%, 60%, 70%, 80%, 90%, or 100% polyester. In some embodiments, textiles are textile waste products. In some embodiments, textiles are made from one or more of clothing, carpets, towels, blankets, sheets, tablecloths, ropes, bags, other textile-based consumer goods, or other textile-based industrial materials.

The size and form of textile may vary according to various embodiments. In some embodiments, textiles include intact textiles. In some embodiments, textiles include textile fibers, also referred to as textile fiber particles. In some embodiments, textiles include a combination of intact textiles and textile fibers.

Textile fibers comprise fibers that are substantially loose or unattached from other fibers. Textile fibers may take the form of a mass of individual fibers that are not uniformly interconnected. For example, textile fibers are often found tangled or amassed with other textile fibers in a non-uniform way. Structurally, textile fibers often have very high aspect ratios. In some embodiments, textile fibers have width to length aspect ratios of greater than 1:20. In some embodiments, textile fibers exhibit different physical properties than intact textiles made from the same material, being generally characterized as exhibiting one or more of lower density, more irregular porosity, lower tensile strength, and lower flexural strength. In some embodiments, separated textile fibers may be produced by shredding, garneting, or detaching textile fibers from intact textiles. In some embodiments, textile fibers include small sections of intact textiles that exhibit physical properties more akin to textile fibers than intact textiles. In some embodiments, textile fibers comprise one or more of lint, thread, yarn, or hair.

An intact textile comprises a multiplicity of textile fibers that are substantially or uniformly woven, knitted, interconnected, or otherwise bonded together to form a discrete unit. In some embodiments, intact textiles exhibit different physical properties than textile fibers made from the same material, being generally characterized as exhibiting one or more of higher density, more regular porosity, higher tensile strength, and higher flexural strength. In some embodiments, intact textiles have width to length aspect ratios of less than 1:20. In some embodiments, intact textiles have a size (length and width) approximately equal to or greater than the size (length and width) of the container (if applicable).

In some embodiments, the substrate does not include any dyes. In some embodiments, the substrate includes one or more dyes. In some embodiments, the one or more dyes are digestible by the mycelium. In some embodiments, the one or more dyes are not digestible by the mycelium. In some embodiments, dyes are substantially removed from the substrate prior to applying 130.

In some embodiments, the substrate is sterilized prior to applying 130. Sterilizing 135 a substrate can eliminate or weaken microorganisms within the substrate that may harm or compete with the mycelium for growth in the resulting composite. In some embodiments, sterilizing 135 includes one or more of heat and pressure treatment. In some embodiments, sterilizing 135 includes one or more of autoclaving, pressure cooking, oven heating, and hot water bathing the substrate.

Providing mycelium 120 involves providing mycelium cells. Mycelium is the vegetative part of a fungus, characterized by a network of mycelial cells called hyphae. In some embodiments, providing mycelium 120 involves providing mushroom spawn, a substrate that has been inoculated with mycelium or has mycelium growing on it. In some embodiments, mushroom spawn substrates include grain, agar, wood chips, sawdust, wood dowels, textiles, or other organic fibrous materials. In some embodiments, providing mycelium 120 involves providing liquid spawn, a liquid solution including mycelial cells and one or more nutrients. In some embodiments, liquid spawn includes one or more of the following components: water, sugar, peptone, agar, malt extract,

Optionally, prior to providing mycelium 120, a substrate may be inoculated with mycelium and incubated to form mushroom spawn. In some embodiments, one or more of the following grains are used as mushroom spawn substrates: sorghum, millet, rye, wheat, rice, corn barley, oats. In some embodiments, inoculating involves adding to the grain mycelium and one or more of water, malt extract, agar, and yeast. In some embodiments, incubating involves maintaining adequate temperature, humidity, and oxygen level for sufficient time to allow mycelial growth on the mushroom spawn substrate. In some embodiments, incubating includes maintaining the inoculated mushroom spawn substrate at approximately, at least, or between any two of 15, 20, 25, 30, 35, or 40° C. In some embodiments, incubating includes maintaining the inoculated mushroom spawn substrate at approximately, at least, or between any two of 50, 60, 70, 80, 90, 99, or 100% relative humidity. In some embodiments, incubating includes maintaining the inoculated mushroom spawn substrate at approximately, at least, or between any two of 10, 15, 20, or 25% oxygen. In some embodiments, incubating includes maintaining the inoculated mushroom spawn substrate at approximate atmospheric levels of oxygen. In some embodiments, incubating includes maintaining one or more of the aforementioned properties for at least 1, 2, 3, 4, 5, 6, 7, 8, 9, 10, 15, 20, 25, 30, 35, 40, 45, or 50 days. In particular embodiments, incubating includes maintaining the inoculated mushroom spawn substrate at approximately 21° C., 80% relative humidity, and atmospheric oxygen levels for at least 10 days.

In some embodiments, the mycelium is non-toxic and non-pathogenic. In some embodiments, the spores, hyphae, and fruiting bodies produced by mycelium are non-toxic and non-pathogenic. In some embodiments, the mycelium is saprotrophic, meaning it lives and feeds on dead organic matter. In some embodiments, the mycelium forms dense, fibrous mycelial networks. In some embodiments, the mycelium consumes and grows on cellulosic materials. In some embodiments, the mycelium consumes and grows on lignocellulosic materials. In some embodiments, the mycelium breaks down and consumes polyesters. In some embodiments, the mycelium breaks down and consumes polyolefins.

In some embodiments, only one species of fungus makes up the mycelium. In some embodiments, more than one species of fungus make up the mycelium. In some embodiments, the mycelium is of genus Pleurotus. In some embodiments, the mycelium is of species Pleurotus ostreatus. In some embodiments, the mycelium is of genus Ganoderma. In some embodiments, the mycelium is of species Ganoderma lucidum. In some embodiments, the mycelium is of species Ganoderma gibbosum.

Applying 130 involves applying the mycelium to the substrate to form a composite. In some embodiments, applying involves placing the mycelium and the substrate into a solid container sufficient for the mycelium and the substrate to contact one another. Such containers may act like molds used to form biocomposites of defined shapes. In some embodiments, containers are shaped like cubes, cuboids, pyramids, prisms, platonic solids, spheres, hemispheres, cones, cylinders, other polyhedral, or other non-polyhedric shapes. In some embodiments, containers are shaped as complex shapes to allow resulting mycelium biocomposites to be used as structural or functional elements in many various applications.

In some embodiments, the ratio of substrate to the mycelium is roughly equivalent. In some embodiments, the amount of substrate in the composite is about, or between any two of 45, 50, 55, 60, 65, 70, or 75% (w/w). In some embodiments, the amount of the mycelium in the composite is at least, about, or between any two of 25, 30, 35, 40, 45, 50, or 55% (w/w).

In some embodiments, the mycelium and substrate are applied in such a manner as to increase contact surface area between the two components. Increased contact surface area between the mycelium and substrate is important for improving mycelium's ability to efficiently colonize substrate material while allowing the mycelium to grow 140 and more quickly result in a functional biocomposite.

In some embodiments, applying involves layering substrate and the mycelium once or more times to create a layered composite. Layering involves 1) placing some amount of substrate on a surface, 2) placing some amount of the mycelium on the placed substrate, and 3) repeating steps 1) and 2) any number of times in not necessarily the same number or amount as one another. For example, layering may include placing substrate and placing the mycelium. In another example, layering involves placing substrate, placing the mycelium, placing substrate, placing the mycelium, and placing substrate. In some embodiments, layering involves placing at least, equal to, or between any two of 1, 2, 3, 4, 5, 6, 7, 8, 9, or 10 layers of substrate. In some embodiments, layering involves placing at least, equal to, or between any two of 1, 2, 3, 4, 5, 6, 7, 8, 9, or 10 layers of the mycelium. In a specific embodiment, applying involves layering substrate and the mycelium into four alternating layers of substrate and the mycelium. Layering may be employed in embodiments using intact textiles to maximize surface area contact between substrate and the mycelium and improve uniformity and flexural properties of the resulting biocomposite. In some embodiments, a single textile is folded according to the aforementioned layering process, wherein each textile layer is a distinct but connected section of the overall textile. In some embodiments, more than one textile are folded according to the aforementioned layering process, wherein at least two layers are made from the same textile. Layering may also be employed in embodiments using a combination of intact textiles and separated textile fibers.

In some embodiments, applying involves combining the mycelium and substrate together. Combining may involve combining the mycelium and substrate to form a heterogenous mixture. In some embodiments, combining involves mixing the mycelium and substrate to increase surface area contact between the two components. Combining may be employed to maximize surface area contact between substrate and the mycelium and improve uniformity and flexural properties of the resulting biocomposite.

Optionally, the composite may be compressed after or concurrent to applying 130 but before allowing mycelium to grow 140. In some embodiments, the composite is compressed for the duration of allowing the mycelium to grow. In some embodiments, the composite is only briefly compressed prior to allowing the mycelium to grow. In some embodiments, the composite is compressed for a duration of at least, equal to, or between any two of 1, 2, 3, 4, 5, 10, 15, 20, 30, or 60 minutes. In some embodiments, the composite is compressed with pressure of at least, equal to, or between any two of 5, 10, 15, 20, 25, or 30 PSI. In some embodiments, the composite is compressed manually, using a compactor, using a mechanical press, or using another like tool for compression. Compressed composites generally exhibit higher density compared to uncompressed composites and thereby have lower oxygen availability to inner mycelial cells. Because oxygen amount is a factor in the growth of fungal fruiting bodies, reducing oxygen availability may reduce likelihood or prevent fruiting body growth out of the mycelium. Prevention of fruiting body growth allows greater uniformity in biocomposite shape and mitigates the potential health risks to humans and other animals caused by spore release from fruiting bodies.

Allowing the mycelium to grow 140 involves allowing the mycelium to establish one or more networks of mycelial cells or hyphae throughout the composite to form a mycelium biocomposite. Mycelial cells grow on a substrate by extending branching filaments called hyphae. In some embodiments, hyphae extend through pores in substrates to provide increased structural stability and bond mycelium to substrate. In some embodiments, mycelium establishes one or more networks through more than section of substrate which has the effect of bonding substrate sections together. In some embodiments, mycelium disposed throughout the composite grow together to form a single network of mycelial cells. In some embodiments, mycelium disposed throughout the composite grow in separate areas of the composite to form more than one networks of mycelial cells. In some embodiments, allowing the mycelium to grow 140 includes incubating the composite. Incubating includes providing favorable conditions to induce mycelial growth in the composite. Among the conditions relevant to incubation are temperature, humidity, access to oxygen, and duration. Each of these factors vary depending on the species of fungus used.

In some embodiments, mycelial cells produced during allowing the mycelium to grow 140 comprise morphologically beneficial compounds including one or more of chitin, chitosan, β-glucan based oligosaccharides, and many others. In some embodiments, mycelial cells provide the biocomposite with beneficial morphological, adhesive, and fire-resistant properties.

In some embodiments, allowing the mycelium to grow 140 includes keeping the composite at a constant temperature. In some embodiments, the composite is kept a constant temperature of at least, equal to, or between any two of 15, 20, 25, 30, 35, or 40° C. In some embodiments, allowing the mycelium to grow 140 includes varying the temperature around the composite. In some embodiments, the temperature around the composite is varied between temperatures of at least, equal to, or between any two of 15, 20, 25, 30, 35, or 40° C. In particular embodiments using the fungus Pleurotus ostreatus, allowing the mycelium to grow 140 includes keeping the composite at a constant temperature of approximately 21° C.

In some embodiments, allowing the mycelium to grow 140 includes keeping the composite at a constant relative humidity. In some embodiments, the composite is kept at a constant relative humidity of at least, equal to, or between any two of 50, 60, 70, 80, 90, 99, or 100%. In some embodiments, allowing the mycelium to grow 140 includes varying the relative humidity around the composite. In some embodiments, the relative humidity around the composite is varied between values of at least, equal to, or between any two of 50, 60, 70, 80, 90, 99, or 100%. In particular embodiments using the fungus Pleurotus ostreatus, allowing the mycelium to grow 140 includes keeping the composite at a relative humidity of approximately 80%.

Fungi may be aerobic or anaerobic which determines whether a low or high certain oxygen level aids in promoting mycelial growth. In some embodiments, allowing the mycelium to grow 140 includes maintaining the composite at a constant oxygen level. In some embodiments, the composite is maintained at approximately, at least, or between any two of 10, 15, 20, or 25% oxygen or the environmental concentration of oxygen at sea level. In some embodiments, particular oxygen levels are maintained by storing composites in open air, in an open-top container, or in a closed-top container with one or more openings in an oxygen-controlled environment. In some embodiments, particular oxygen levels are maintained by storing composites in a container with controlled oxygen levels. In some embodiments, environmental oxygen level is maintained by storing composites in open air, in an open-top container, or in a closed-top container with one or more openings. In particular embodiments using the aerobic fungus Pleurotus ostreatus, allowing the mycelium to grow 140 includes maintaining the composite at the environmental concentration of oxygen at sea level.

Mycelium colonization rates may depend on fungus species, substrate digestibility, temperature, relative humidity, and oxygen level, among other factors. In some embodiments, allowing the mycelium to grow 140 lasts for at least, equal to, or between any two of 5, 10, 15, 20, 25, 30, 35, 40, 45, 50, or 100 days. In some embodiments, allowing the mycelium to grow 140 lasts for as long as it takes to achieve a particular colonization % throughout the substrate. In some embodiments, allowing the mycelium to grow 140 lasts for as long as it takes to achieve at least, equal to, or between any two of 20, 30, 40, 50, 60, 70, 80, 90, 99, or 100% colonization. In some embodiments, colonization % may be measured by determining what percentage of cubic units of the biocomposite mycelium has spread to.

In particular embodiments using Pleurotus ostreatus fungus, cotton or polyester substrates, 21° C. temperature, and 80% relative humidity, allowing the mycelium to grow may last for approximately 21 days.

Composites may also reduce in mass over the course of allowing the mycelium to grow. In some embodiments, the mass of the composite compared to the resulting biocomposite shrinks at least, equal to, or between any two of 5, 10, 15, 20, 25, 30, 35, and 40%. Overall, as the proportion of mycelium to substrate decreases, the degree of shrinkage also declines. This is likely due to smaller initial mycelium colonies having less capacity to metabolizing substrate than larger colonies.

Optionally, mycelial growth may be arrested by changing one or more of the temperature, humidity, and oxygen levels. In some embodiments, mycelial growth is arrested by removing at least, equal to, or between any two of 50, 60, 70, 80, 90, 99, or 100% of all moisture in the mycelium-based biocomposite. In some embodiments, mycelial growth is arrested by raising or lowering the temperature outside of the range of optimal growth. In some embodiments, mycelial growth is arrested by raising the temperature of the mycelium-based biocomposite to at least, equal to, or between any two of 35, 40, 45, 50, or 55° C. In some embodiments, mycelial growth is arrested by lowering the temperature of the mycelium-based biocomposite to less than, equal to, or between any two of 10, 5, 0, −5, or −10° C. In some embodiments, mycelial growth is arrested by moving the mycelium-based biocomposite to an environment with less than, equal to, or between any two of 15, 10, 5, or 0% oxygen.

Mycelium-based biocomposites have various beneficial properties. In some embodiments, mycelium is integrated into the substrate. In some embodiments, mycelium-based biocomposites have mycelial growth throughout the substrate material. In some embodiments, mycelium-based biocomposites have mycelial growth across at least, equal to, or between any two of 10, 20, 30, 40, 50, 60, 70, 80, 90, or 99% of the biocomposite's volume. In some embodiments, mycelium-based biocomposites have mycelial growth covering at least, equal to, or between any two of 10, 20, 30, 40, 50, 60, 70, 80, 90, or 99% of the pores of the biocomposite. In some embodiments, mycelium-based biocomposites grow throughout the substrate and consume at least part of the substrate. In some embodiments, mycelium-based biocomposites comprise mycelium of at least, equal to, or between any two of 25, 30, 35, 40, 45, 50, 55, 60, 65, 70, 75, 80% (w/w). In some embodiments, mycelium-based biocomposites have mycelial growth dense and widespread enough to prevent other fungus from growing on the substrate.

In some embodiments, mycelium-based biocomposites using only intact textiles as the substrate (hereafter referred to as “intact biocomposites”) exhibit enhanced morphological, mechanical, and flammability-related properties compared to mycelium-based biocomposites using only separated textile fibers as the substrate (hereafter referred to as “fiber biocomposites”). In embodiments using some combination of intact textiles and separated textile fibers (hereafter referred to as “combined biocomposites”), some of these properties improve along with the proportion of intact textiles making up the substrate and improve inversely with the number of unconnected intact textiles. Some improvements may be attributed to the inherent properties of intact textiles compared to separated textile fibers. For example, a biocomposite made with 1 intact textile has improved morphological properties compared to a biocomposite of the same size made from 10 disconnected intact textiles as well as a biocomposite of the same size made from separated textile fibers. In some embodiments, the enhanced properties of intact biocomposites may be attributed to the intact textiles providing a denser substrate for mycelium to grow upon. In some embodiments, the mycelium acts as a secondary binder, reinforcing the already cohesive textile matrix. In some embodiments, the mycelium serves as the primary binder, resulting in weaker overall structural integrity due to the lack of pre-existing connections between substrate components.

In some embodiments, mycelium-based biocomposites exhibit excellent flexural strength. In some embodiments, intact biocomposites exhibit superior flexural strength compared to fiber biocomposites and combined biocomposites made with similar materials. In some embodiments, mycelium-based biocomposites exhibit flexural strength of at least, equal to, or between any two of 5, 10, 20, 30, 40, 50, 60, 70, 80, 90, 100, 110, 120, and 130 kPA.

In some embodiments, mycelium-based biocomposites exhibit enhanced ultimate strain properties. Ultimate strain refers to the maximum flexural strain a material can withstand before failure, as measured during flexural testing. It is calculated based on the curvature of the sample at the point of fracture, specifically at the outermost surface of the material. A higher ultimate strain value indicates greater flexibility and the ability of the biocomposite to deform more extensively under bending stress before breaking. In some embodiments, mycelium-based biocomposites exhibit ultimate strain of at least, equal to, or between any two of 20%, 30%, 40%, 50%, 60%, or 70%.

In some embodiments, mycelium-based biocomposites exhibit enhanced toughness. In some embodiments, mycelium-based biocomposites exhibit toughness of at least, equal to, or between any two of 100, 200, 300, 400, 500, 1000, 1500, 2000, 3000, and 3500 kJ/m³.

These morphological and mechanical properties demonstrate that mycelium-based biocomposites are well-suited for a wide range of applications that require sturdy materials such as building materials, packaging, furniture, and clothing.

In some embodiments, mycelium-based biocomposites have low flammability. In some embodiments, thin portions (less than 5 mm thickness) of mycelium-based biocomposites when ignited burn less than, equal to, or between any two of 80, 70, 60, 50, 40, and 30% over the period of one minute. In some embodiments, thin portions (less than 5 mm thickness) of mycelium-based biocomposites exhibit combustion rates of less than, equal to, or between any two of 0.8, 0.7, 0.6, 0.5, 0.4, 0.3, 0.2, 0.1, or 0.01 cm/s while burning. In some embodiments, mycelium biocomposites exhibit low average and peak heat release rates. In some embodiments, mycelium-based biocomposites exhibit low flashover times. The reduced overall combustibility of mycelium-based biocomposites may be attributed to their higher charring tendency which functions as a thermal insulator and restricts the availability of combustible gases to the flame front.

In some embodiments, mycelium-based biocomposites have comparable thermal stability to textiles made from the same material as the substrate. In some embodiments, mycelium-based biocomposites have comparable rates of weight change under thermogravimetric analysis (TGA). In some embodiments, mycelium has little impact on thermal stability and rate of weight change under TGA of mycelium-based biocomposites. In some embodiments, mycelium-based biocomposites containing cotton exhibit primary weight loss from TGA at 300-380° C. In some embodiments, mycelium-based biocomposites containing polyester exhibit primary weight loss from TGA at 330-660° C.

In some embodiments, mycelium-based biocomposites have high humidity absorption properties. In some embodiments, mycelium-based biocomposites have improved humidity absorption properties compared to textiles made from the same material as the substrate. In some embodiments, mycelium-based biocomposites made with cotton substrates exhibit at least, equal to, or between any two of 2, 3, 4, 5, 6, 7, and 8% humidity absorption at 65% relative humidity and 21° C. In some embodiments, mycelium-based made with cotton substrates biocomposites exhibit at least, equal to, or between any two of 2, 3, 4, 5, 6, 7, 8, 9, 10, 11, 12, 13, and 14% humidity absorption at 90% relative humidity and 21° C. In some embodiments, mycelium-based biocomposites made with polyester substrates exhibit at least, equal to, or between any two of 1, 2, 3, 4, and 5% humidity absorption at 65% relative humidity and 21° C. In some embodiments, mycelium-based biocomposites made with polyester substrates exhibit at least, equal to, or between any two of 1, 2, 3, 4, 5, and 6% humidity absorption at 90% relative humidity and 21° C. The trend of biocomposites exhibiting improved humidity absorption capabilities compared to textiles might be explained by biocomposites having an increased surface area compared to textiles.

In some embodiments, mycelium-based biocomposites are not hydrophobic. In some embodiments, mycelium-based biocomposites have water contact angles of approximately or between any two of 10, 130, 140, and 150°. In some embodiments, mycelium-based biocomposites have water contact angles approximately equivalent to textiles made from the same material as the substrate. These are true despite mycelium often exhibiting highly hydrophobic properties. As a result, it is likely that the growth of mycelium does not greatly affect the hydrophobicity of mycelium-based biocomposites.

In some embodiments, mycelium-based biocomposites exhibit sound dampening properties. These properties may be attributed to mycelium-based biocomposites having highly porous structure or low densities in embodiments that feature one or both of those properties.

In some embodiments, mycelium-based biocomposites require lower energy consumption than comparable construction materials. In some embodiments, mycelium-based biocomposites exhibit lower material criticality than comparable construction materials such as polyurethane foam. In some embodiments mycelium-based biocomposites exhibit lower environmental impacts regarding one or more of acidification, freshwater eutrophication, human carcinogenicity, and particulate matter created than comparable construction materials such as polyurethane foam and polystyrene foam.

Any aspects of any embodiment and/or example may be utilized across any and all embodiments of the present invention.

### EXAMPLES

The following experiments were carried out using three replicates: mechanics, shrinkage, and water absorption. The flammability analysis was conducted using eight replicates. In these examples, the results are expressed as mean±standard deviation. Statistical analyses were conducted, and data visualization was performed using Origin 2023B.

Example 1. Culture preparation. The fungal strain used in this and subsequent examples was Pleurotus ostreatus (oyster mushroom, variety: florida), obtained commercially as a cultivated grain spawn bag. The following textile materials were tested herein: 100% cotton and 100% polyester. The procured textile materials were washed with deionized water (21° C.) to remove dust and loose textile threads, followed by drying overnight at 50° C. Agar, yeast extract sourced from Glenthem Life Sciences, and malt extract from commercial sources (Craft a Brew) were utilized as received for mycelium propagation and culture. The obtained strain of P. ostreatus was cultured in malt agar medium prior to its use in bio block preparation. The medium was prepared by adding 10 g malt extract, 10 g agar, and 1 g yeast extract to 500 mL deionized water, followed by sterilization in an autoclave (Rodwell autoclave phoenix 60) at 121° C. and 2 atm for 15 min. After cooling down to 50° C., 25 mL of agar media was poured into sterile petri dishes, followed by 10 mins cooling in a biosafety cabinet to solidify. The agar plates were then inoculated using commercially obtained grain spawns utilizing sterile forceps. The inoculated dishes were incubated for 10 days at 21° C. and 80% relative humidity. After culture cultivation, overnight hydrated sorghum grains were prepared in a cylindrical grow bag (20 cm diameter, 47 cm height). The grains were autoclaved and later inoculated with the fully grown mycelium agar plates. The inoculated grains were incubated at 21° C. and 80% relative humidity for 14 days.

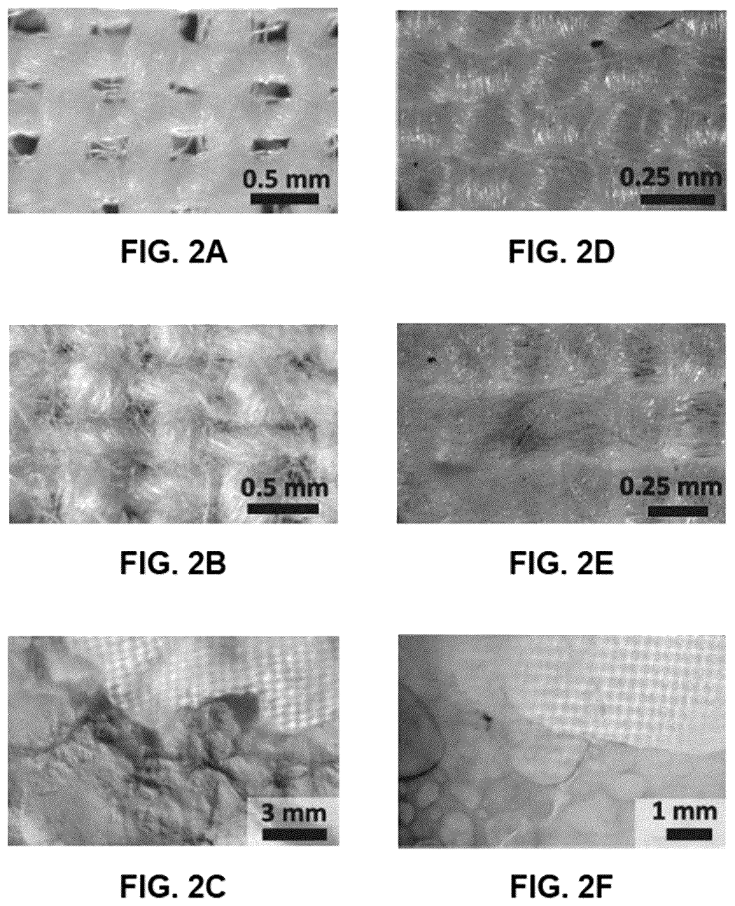

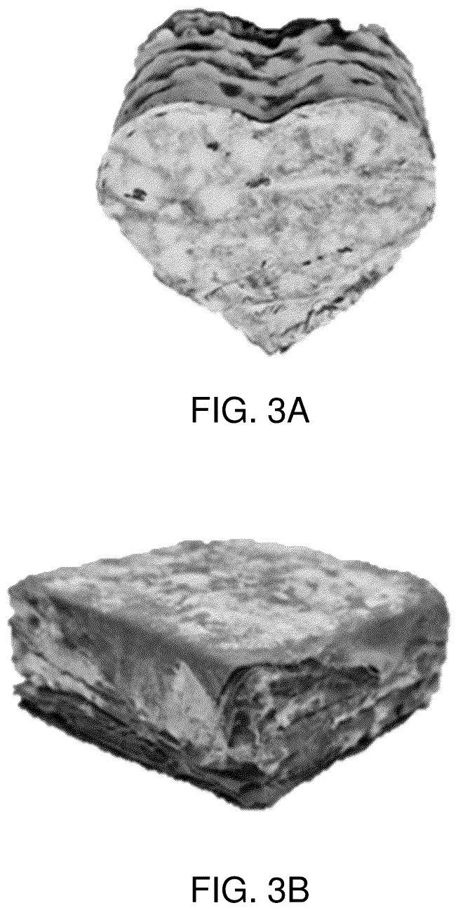

Example 2. Biocomposite preparation (layered biocomposites). Mycelium-based biocomposites were prepared using the following procedure. Textiles were cut into 11.43 cm×3.81 cm strips, followed by washing and drying. FIGS. 2A and 2D illustrate light microscopy images of intact textiles made from 100% cotton and 100% polyester, respectively. A mold made from silicon (dimensions: 11.43 cm×3.81 cm×2.54 cm) was procured. The textile strips and the mold were sterilized using an autoclave.

Four layers of textile samples and four layers of grain spawns were placed alternatingly in the mold. Different mass ratios of cotton (CO) or polyester (PES) to grain spawn were utilized as a matrix for the biocomposite preparation: 50% cotton 50% spawn (5050CO), 60% cotton 40% spawn (6040CO), 70% cotton 30% spawn (7030CO), 50% polyester 50% spawn (5050PES), 60% polyester 40% spawn (6040PES), and 70% polyester 30% spawn (7030PES). Amount of spawn added varied based on the relative ratios of textile to spawn. For instance, in 50:50 samples, 3.75 g of textile and 3.75 g of grain spawn were used. A final layer of textile was placed on the top to reduce possible contamination, and the mold was further covered by two layers of cling wrap (Falcon) with 5 holes pocked by a syringe for airflow. Eight samples were prepared for each textile type, and the mold was incubated for 21 days at 80% relative humidity and 21° C. The growth was subsequently terminated by drying for 24 hours at 50° C. FIGS. 2B and 2C illustrate light microscopy images of 5050CO biocomposite at various zoom levels. FIGS. 2E and 2F illustrate light microscopy images of 5050PES biocomposite at various zoom levels.

After the 21-day incubation period, colonization of the textile matrix appeared to depend on the concentration of spawn within the layered textiles. Analysis of the cotton samples indicates the presence of non-colonized areas 215 in the 7030CO (Textile: Grain ratio—7030, CO—Cotton textile) and 6040CO biocomposites. These areas of non-colonization appeared to diminish in size as the textile concentration decreased and the spawn concentration increased, with nearly complete colonization seen in the 5050CO samples. Notably, dense areas of surface growth were observed, particularly in the 5050CO sample, whereas this growth was less pronounced in the 6040CO and 7030CO samples. Additionally, the 5050CO samples demonstrated structural integrity, maintaining their shape and stability when positioned along the shortest edge of the biocomposite.

Other samples were made with the aforementioned procedure using low-quality non-woven cotton fibers. These biocomposites resulted in soft, foamy, and flexible structures, exhibiting the highest water absorption compared to other substrates.

Growth patterns observed in the polyester samples differed significantly from those in cotton samples. All polyester-based matrices exhibited colonization without any non-growth zones. As observed with the cotton fabric, increased spawn concentrations similarly enhanced growth in the polyester fabrics, with the 5050PES samples exhibiting the highest growth. However, dense growth zones found in 5050CO were not evident on the surface of the polyester samples. This difference is likely due to the nature of the textile fibers because natural fibers like cotton offer a more accessible carbon source for mycelial growth whereas synthetic fibers such as polyester are less readily metabolized by the mycelium. The limited nutrient availability in polyester fabrics may prompt the mycelium to adopt a foraging strategy, producing fast-growing, elongated branches with loose aggregations as it searches for nutrients. This pattern may explain the comparatively higher overall colonization in polyester samples, although the weaving pattern is likely to play a role as well. Conversely, the cotton samples support the mycelium's ability to utilize and degrade the fibers, leading to a “phalanx” growth mode characterized by slower expansion but dense aggregation. This mode likely accounts for the dense surface growth and relatively lower colonization observed in cotton samples at lower spawn concentrations.

Biocomposites were also prepared using molds other than rectangular shapes, including square and heart shapes, and were constructed with multiple layers of spawn, also testing the effect of various fabric finish and color. Spawn growth followed a complex contour with acute angles and allowed folded fabric to adhere. FIG. 3A illustrates a photograph of a heart-shaped mycelium-based biocomposite, according to some embodiments. FIG. 3B illustrates a photograph of a cuboid-shaped mycelium-based biocomposite, according to some embodiments.

Example 3. Biocomposite preparation (mixed biocomposites). Furthermore, separated textile fibers were investigated as a potential alternative substrate for mycelium growth. Biocomposites were fabricated with mixed shredded fibers obtained as lint with a lint-to-spawn ratio 50:50 by volume, demonstrating good colonization even after just one week of incubation under conditions similar to those of the textile composites. The tested mycelium exhibited the capacity to bind to the lint fibers, resulting in a composite with improved structural integrity due to mycelium adhesion. In contrast, the dry lint fibers demonstrated no adhesion between the fibers when evaluated independently. Alternatively, liquid spawn was tested for mycelium growth between textiles instead of grain spawn. The resulting biocomposite exhibited adhesion between the cotton layers, but this adhesion was significantly weaker compared to that achieved with grain spawn biocomposites.

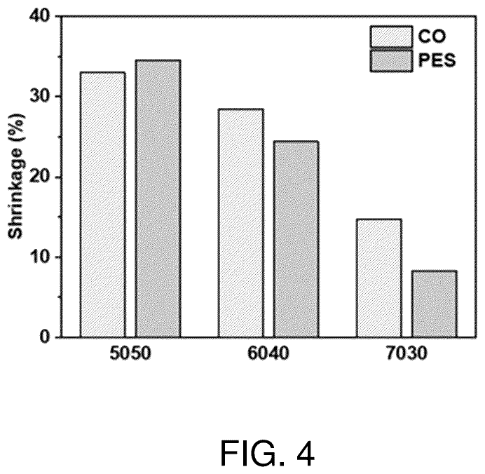

Example 4. Shrinkage analysis. The shrinkage in the overall weight of the biocomposites after two weeks of growth and subsequent 24 hr drying was determined by measuring the weights at those specific intervals using a digital balance (Ohaus Pioneer Precision Balance PA323). The mass loss or “shrinkage” analysis of the biocomposites showed that those containing 50% textile exhibited the highest shrinkage, with reductions of approximately 30-35% observed in both cotton and polyester samples. As the proportion of spawn decreased, the degree of shrinkage also declined, with the lowest shrinkage observed in the 70% textile samples, ranging from 8-12%. FIG. 4 illustrates a graph showing results of the foregoing shrinkage analysis regarding various biocomposites.

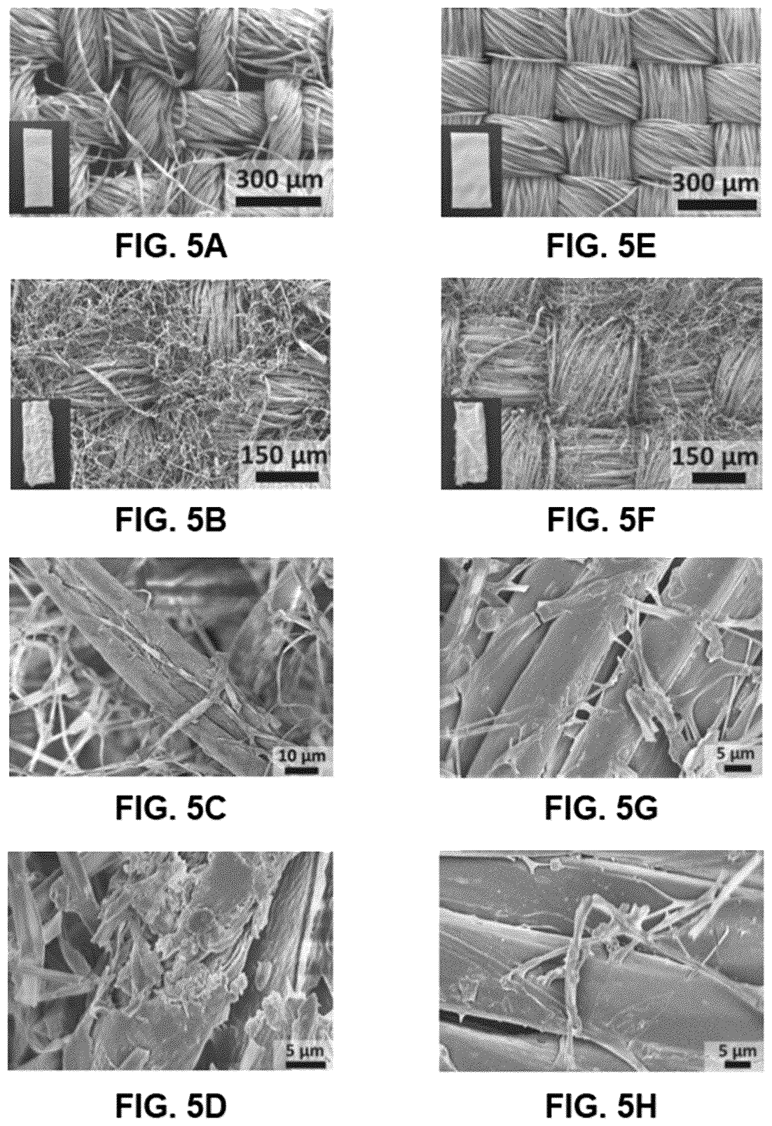

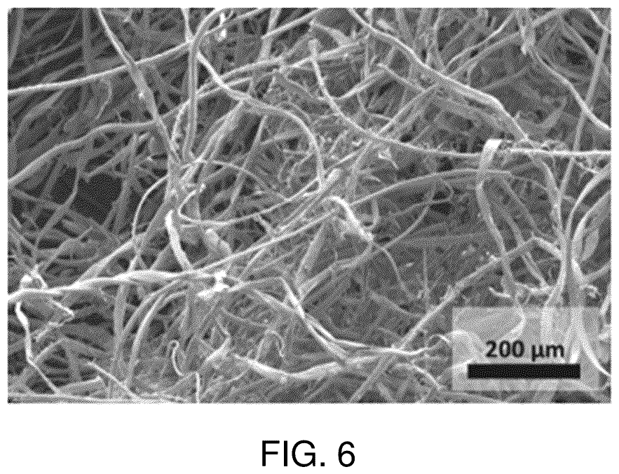

Example 5. Morphological analysis. The morphology of textile and textile-mycelium composite was observed using optical (Leica M165 FC) and scanning electron microscopy (SEM JEOL JSM-7610F). To analyze the growth and propagation of mycelium, the top layer of 50:50 samples in the biocomposites were extracted and dried. For optical microscopy, the samples were analyzed in their native state without modification. For SEM analysis, a 30 second gold coating on the sample was applied using a sputter coater (AUTO fine coater; JEOL JEC-3000FC). Subsequently, analysis was performed at an accelerating voltage of 3 kV with varying magnifications. FIG. 5A illustrates an SEM image of an intact textile made of 100% cotton. FIGS. 5B-5D illustrate SEM images of 5050CO biocomposites at varying zoom levels. FIG. 5E illustrates an SEM image of an intact textile made of 100% polyester. FIGS. 5F-5H illustrate SEM images of 5050PES biocomposites at varying zoom levels. FIG. 6 illustrates an SEM image of a biocomposite made using separated textile fibers made from lint as a substrate.

Morphological observations of the pure cotton textile samples revealed a porous, fibrous structure with fiber diameters of ˜0.35 mm, along with rectangular pores between the horizontally and vertically arranged fibers. In contrast, polyester samples displayed a denser arrangement within the tightly knit fibers of ˜0.25 mm diameter. Images obtained from optical and scanning electron microscopy of the 5050CO samples further validated the previously hypothesized dense packing of fibers in cotton samples. These images revealed substantial growth around the sample, with mycelium effectively covering the pores in the cotton fiber matrix, potentially enhancing its structural robustness. By contrast, the 5050PES samples exhibited loose colonization at the microscopic level. Dense surface growth on the 5050CO samples was also confirmed through microscopy, with a comparatively lower surface growth observed on polyester samples.

The capacity of P. ostreatus to degrade cotton fibers was apparent, as mycelium was observed to partially break down fibers at points of colonization, further suggesting utilization of cotton as a nutrient source. The mycelial fibers also appeared integrated into the cotton fibers to some extent. Furthermore, the mycelium was visibly unable to penetrate or degrade the synthetic polyester fibers. This lack of degradation may be attributed to the enzymes produced by P. ostreatus, which are not highly effective against synthetic fibers. While integration and metabolization of the PES fibers by the mycelium are less clear, their interfacial adhesion is rather evident, highlighting that both the network of mycelium fibers and interfacial adhesion are likely contributors of the mechanical integrity of the biocomposites.

Scanning electron microscopy of lint samples was also conducted, revealing a loose mixture of textile fibers and hair strands with noticeably lower density than either intact textile-based biocomposites.

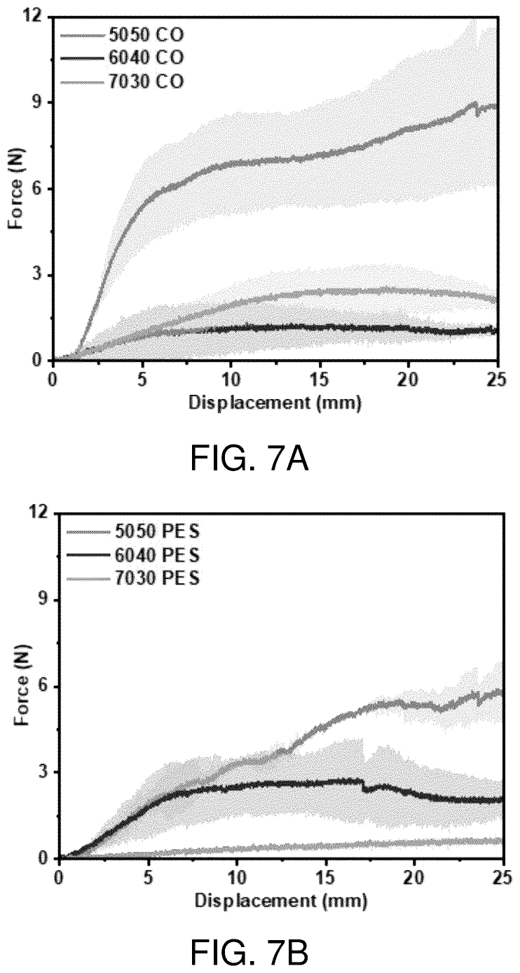

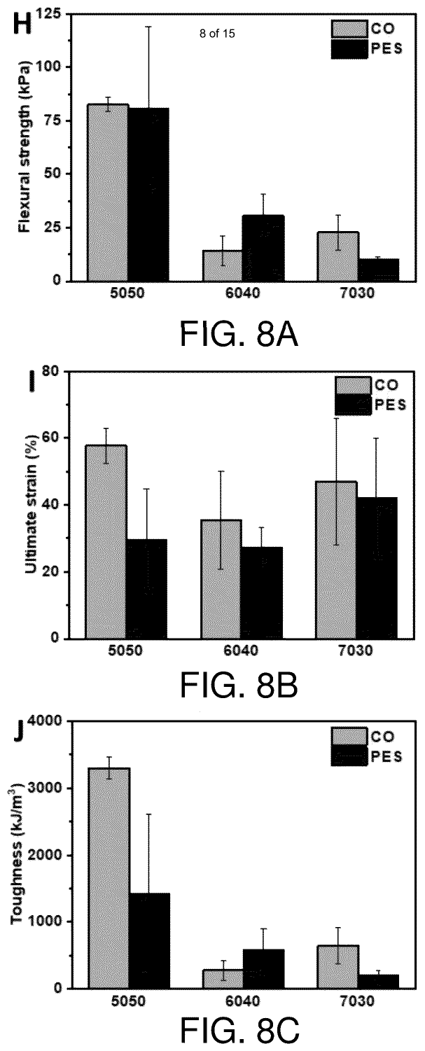

Example 6. Mechanical properties analysis (layered biocomposites). Flexural properties of layered biocomposites were evaluated by a three-point bending test on the prepared samples on a universal testing machine (Instron 5969, Norwood, MA). The load cell had a capacity of 50 kN, and the load rate was set to 0.3 mm/s. The samples had a width of 39 mm and a thickness of 18 mm, with a support span of 70 mm. Each test was conducted in triplicate. FIG. 7A illustrates a graph showing results of force-displacement analysis for biocomposites 5050CO, 6040CO, and 7030CO. FIG. 7B illustrates a graph showing results of force-displacement analysis for biocomposites 5050PES, 6040PES, and 7030PES. FIG. 8A illustrates a graph showing results of flexural strength analysis for biocomposites 5050CO, 6040CO, 7030CO, 5050PES, 6040PES, and 7030PES. FIG. 8B illustrates a graph showing results of ultimate strain analysis for biocomposites 5050CO, 6040CO, 7030CO, 5050PES, 6040PES, and 7030PES.

Control textile samples of cotton and polyester showed no flexural strength. The 7030CO sample exhibited increased structural integrity and reduced sagging between supports, while the 5050CO sample displayed minimal sagging and is seen as a cohesive and solid block. This effect is attributed to the binding properties of mycelium, wherein interconnected fibers adhere to degraded substrate particles through the formation of adhesive compounds, such as chitin and β-glucan based oligosaccharides. It was observed that slippage occurred during testing when displacement exceeded 25 mm, which relates to slippage, i.e., to non-catastrophic failure of the composites. Analysis of the load-displacement curve revealed that the 5050CO sample exhibited a stiffer response, characterized by a steep initial slope in the curve, whereas the 6040CO and 7030CO samples demonstrated a decrease in stiffness. However, similar stiffness was not observed in the 5050PES samples, which displayed relative lower stiffness compared to the 5050CO samples. Increasing the spawn concentration from 30% to 50% in cotton-based composites resulted in an increase in the mean flexural strength from 22.7 kPa to 82.5 kPa, an increase in mean flexural strain from 47% to 57%, and an increase to mean toughness from 645 kJ/m3 to 3298 kJ/m3. The 5050CO biocomposite exhibited the best overall performance, attributed to its higher interlayer interactions with the textile and thicker fiber growth observed in prior analyses. It has also been reported that cultivation in a cellulose-rich substrate promotes the production of chitin, a rigid polymer, which may contribute to the higher mechanical strength observed in 5050CO. This increase in chitin content might also enhance the ductility of mycelium fibers.

In polyester samples, the flexural strength notably increased from 9.9 kPa to 80.5 kPa with an increase in spawn concentration from 30% to 50%, though there was considerable variability across samples. The ultimate strain was reduced from 42% to 29% as the textile concentration decreased from 70% to 50%. Toughness values in polyester samples were notably lower than in cotton samples; the 5050PES exhibited nearly half the toughness value, at 1414 kJ/m³, compared to the 5050CO, while the 6040PES and 7030PES samples showed lower performance with toughness values of 575 kJ/m³ and 199 kJ/m³, respectively.

Example 7. Mechanical properties analysis (mixed biocomposites). Flexural properties of mixed biocomposites were evaluated by placing the biocomposite between two supports and placing successively more weights in the middle to determine the biocomposite's load-bearing capacity.

The lint biocomposite demonstrated good load-bearing capacity by exhibiting no bending when placed between two supports, even with a 250 g weight on top. In stark contrast, the raw lint blocks displayed no structural integrity, failing to support even 150 g weight.

The biocomposite prepared using liquid spawn demonstrated better mechanical strength and retained its shape when placed between supports, unlike raw cotton samples, which did not retain their shape. However, when a 150 g weight was placed on top, the sample collapsed, indicating low load-bearing capacity.

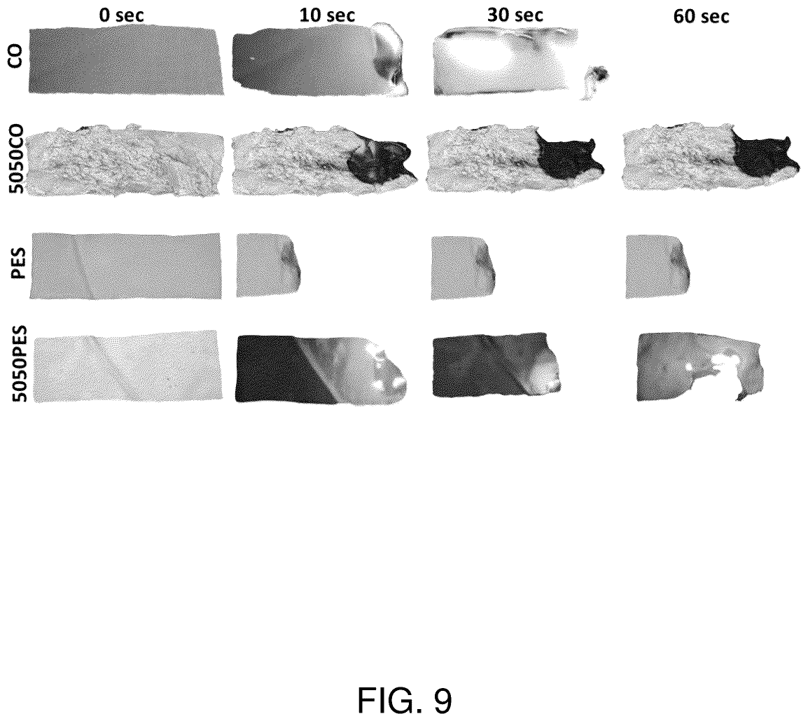

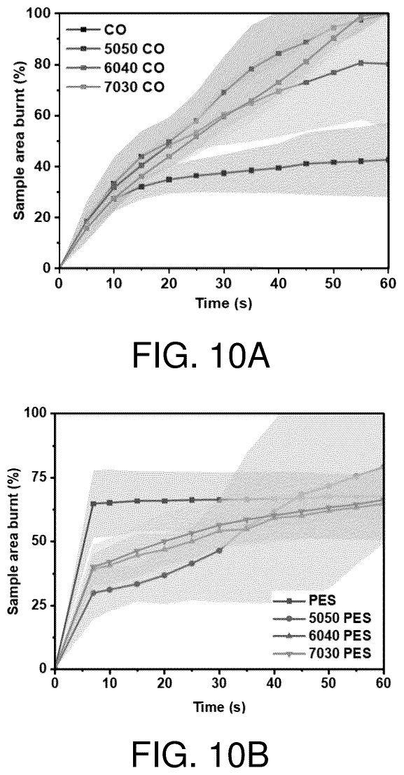

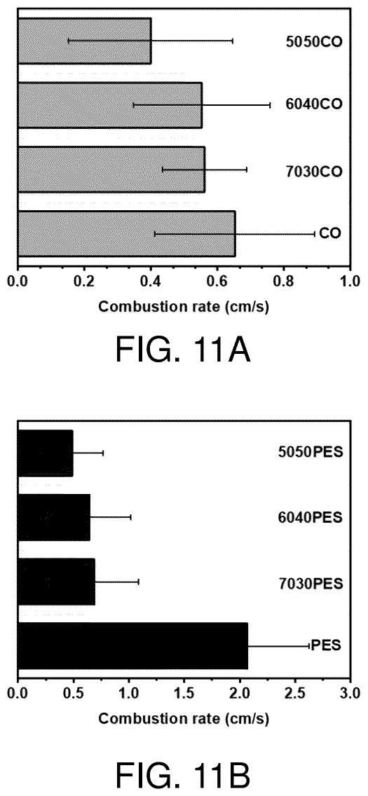

Example 8. Flammability analysis. The rate, extent, and duration of pyrolysis were determined by using protocols from ASTM D635-22 with modifications. The top layer of textile material was extracted from each biocomposite and tested, with a minimum of 5 trials for each sample. The extracted textile material (11.43 cm×3.81 cm) was placed on a flat stage within a controlled environment and ignited at the short edge end for 5 seconds for cotton samples and 7 seconds for polyester samples. A digital camera was used to record the process, and the acquired images and videos were analyzed using ImageJ to calculate the rate, duration, and extent of burning. The flame-retardant properties of the biocomposites were evaluated employing a horizontal combustion test. FIG. 9 illustrates an image array showing photographs taken at 0, 10, 30 and 60 seconds after ignition for control cotton textile, 5050CO, control polyester textile, and 5050PES samples. FIG. 10A illustrates a graph showing % sample area burnt vs time for control cotton textile, 5050CO, 6040CO, and 7030CO. FIG. 10B illustrates a graph showing % sample area burnt vs time for control polyester textile, 5050PES, 6040PES, and 7030PES. FIG. 11A illustrates a bar chart comparing combustion rate (cm/s) of control cotton textile, 5050CO, 6040CO, and 7030CO. FIG. 11B illustrates a bar chart comparing combustion rate (cm/s) of control polyester textile, 5050PES, 6040PES, and 7030PES.

The control cotton textile sample ignited readily, with the flame spreading rapidly across the layer and consuming the material within 60 seconds. In contrast, the mycelium-grown cotton fabric demonstrated notable fire resistance, particularly in the 5050CO samples, which self-extinguished without complete consumption of the fabric. Analysis of the unburned area in the 5050CO sample revealed that fire propagation halted after approximately 30 seconds, leaving only 40% of the total area burned and effectively preventing further flame spread. The superior flame-resistant properties of 5050CO samples compared to other samples may be attributed to the higher growth of mycelium, which contributed to enhanced flame-retardant performance.

Conversely, the control polyester textile exhibited different combustion behavior, wherein it ignited quickly, shrank during burning, but ceased combustion immediately upon removal of the ignition source. These properties may be attributed to polyester being a synthetic fiber. The addition of mycelium growth in 5050PES significantly reduced flame propagation and combustion rates. Nonetheless, the overall burned area remained similar to that of the control polyester samples.

Example 9. Thermogravimetric analysis (TGA). The thermal stability of control cotton textile, control polyester textile, 5050CO, 5050PES, and pure mycelium was investigated using thermogravimetric analysis (NETZSCH High Temperature STA 649 F3 Jupiter). Pure mycelium was grown on sterilized sorghum grains using the same procedure as Example 2, but without adding a textile substrate. The resulting mycelium fibers were then extracted for TGA analysis.

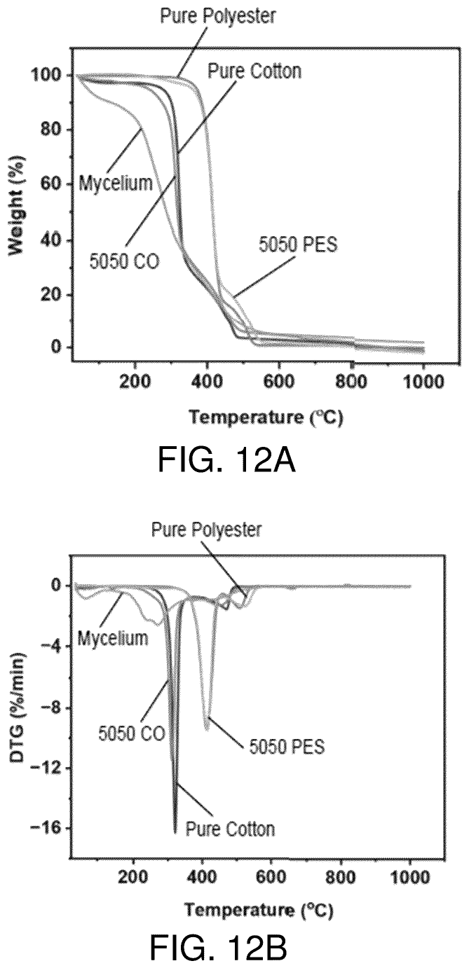

10 mg of sample was placed in an alumina crucible under controlled temperature increase of 5° C./min rate to 1000° C. and air atmosphere. Derivative thermogravimetric analysis (DTG) included calculating the first derivative of the TGA data and was used to determine the rate of weight change for each sample. FIG. 12A illustrates the TGA mass profiles for control cotton textile, control polyester textile, 5050CO, 5050PES, and pure mycelium.

The TGA/DTG mass profiles showed three distinct degradation stages with a small difference between pure cotton and 5050CO, or pure polyester and 5050PES. For cotton-based samples, the main pyrolysis phase (300-380° C.) led to cellulose degradation, producing glucose and other gases. Polyester samples exhibited primary weight loss between 330-660° C. due to polyester breakdown, releasing various polymers and aromatic compounds, with char formation above 500° C. The mycelia fiber's initial degradation (40-220° C.) was due to the volatilization of hydrophobins, while the main mass loss (225-372 650° C.) was due to fungal cell wall decomposition including chitin and other stable components. Although the impact of mycelium fibers is remarkable on the cohesion and flame resistance of the biocomposites, these small differences in TGA curves may indicate that a relatively small amount of mycelium fiber may be present, in absolute.

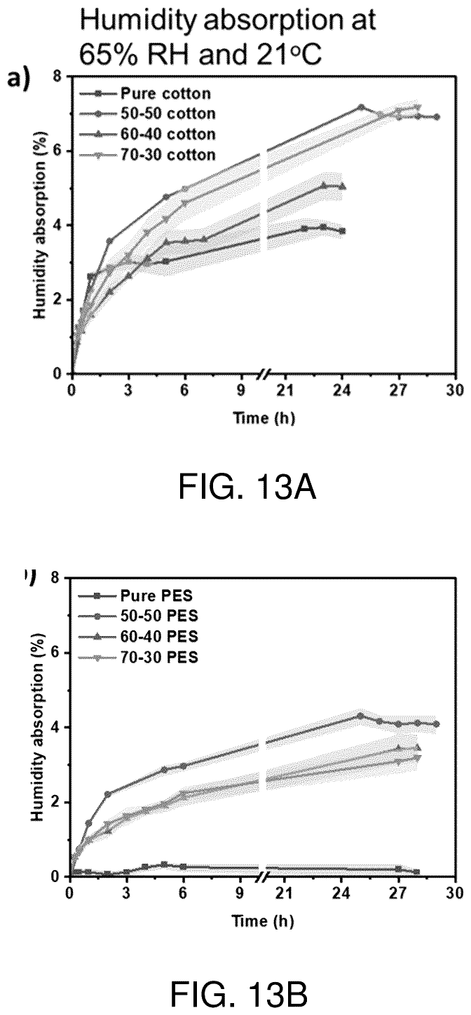

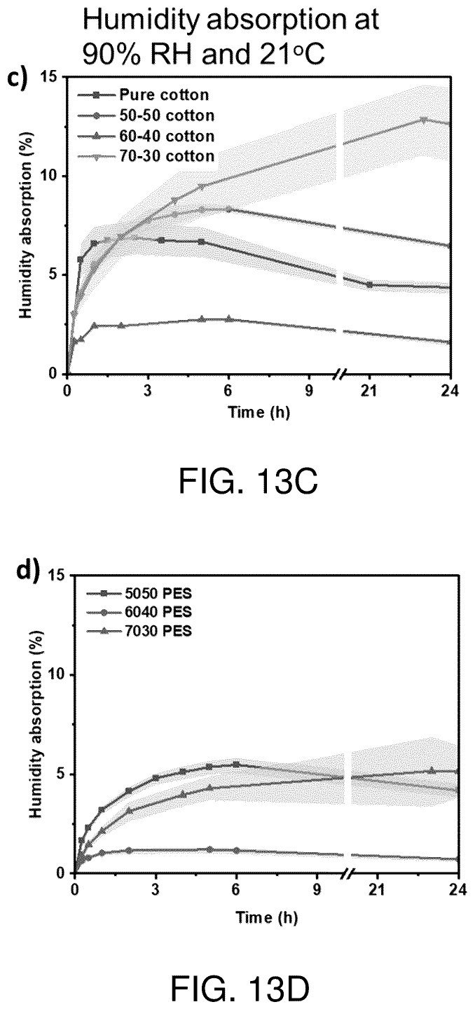

Example 10. Humidity absorption analysis. The humidity absorption test was conducted in accordance with ASTM C272/C272M-16 at 21° C. Samples that had been oven-dried overnight at 50° C. were placed in controlled humidity chambers at 65% relative humidity and 90% relative humidity, and their mass changes were monitored gravimetrically over a period of 24 hours. FIG. 13A illustrates a graph showing the results of humidity absorption analysis conducted at 65% relative humidity (RH) for control cotton textile, 5050CO, 6040CO, and 7030CO. FIG. 13B illustrates a graph showing the results of humidity absorption analysis conducted at 65% relative humidity (RH) for control polyester textile, 5050PES, 6040PES, and 7030PES. FIG. 13C illustrates a graph showing the results of humidity absorption analysis conducted at 90% relative humidity (RH) for control cotton textile, 5050CO, 6040CO, and 7030CO. FIG. 13D illustrates a graph showing the results of humidity absorption analysis conducted at 90% relative humidity (RH) for 5050PES, 6040PES, and 7030PES. The results for both cotton- and polyester-based samples indicated that each biocomposite sample exhibited higher humidity absorption compared to control textile samples at 65% relative humidity. At 90% relative humidity, all cotton-based biocomposites exhibited higher humidity absorption than the control textile sample except for 6040CO. The trend of biocomposites exhibiting improved humidity absorption capabilities might be explained by biocomposites having an increased surface area compared to control textiles.

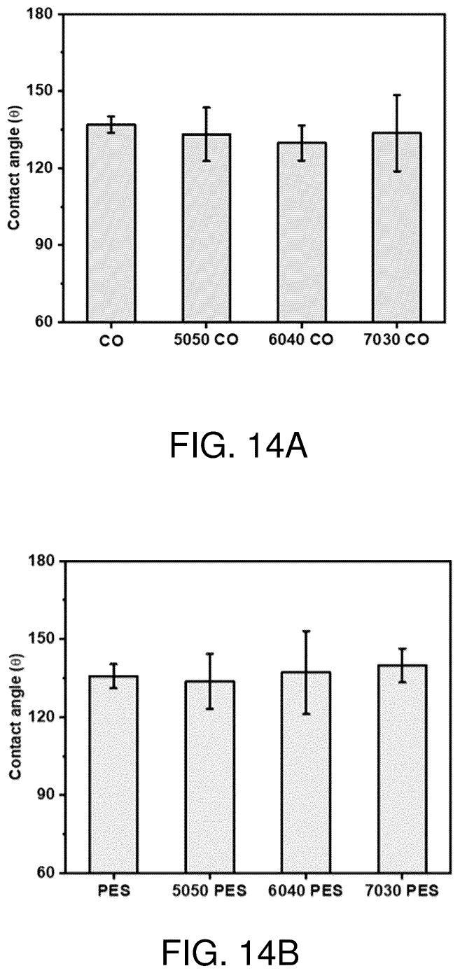

Example 11. Contact angle analysis. The water contact angle of the biocomposites were measured using a goniometer (DSA25, Kruss). Biocomposites were positioned on a stage, and a droplet of 5 μL of deionized water was dropped onto the surface. The contact angle was continuously recorded for a duration of 1 min. FIG. 14A illustrates a bar chart showing contact angle data for control cotton textile, 5050CO, 6040CO, and 7030CO. FIG. 14B illustrates a bar chart showing contact angle data for control polyester textile, 5050PES, 6040PES, and 7030PES. Contact angle measurements revealed that the growth of mycelium did not affect the water affinity characteristics, even though mycelium is inherently highly hydrophobic.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method for making a mycelium-based biocomposite comprising:
providing a substrate including one or more textiles,
providing mycelium,
applying the mycelium to the substrate to form a composite, and
allowing the mycelium to grow to form a mycelium biocomposite.

2. The method of claim 1, wherein the one or more textiles includes intact textiles.

3. The method of claim 1, wherein the one or more textiles includes separated fibers.

4. The method of claim 1, wherein the one or more textiles includes a combination of intact textiles and separated fibers.

5. The method of claim 1, wherein providing mycelium includes providing grain spawn.

6. The method of claim 1, wherein providing mycelium includes providing liquid spawn.

7. The method of claim 1, wherein applying includes placing textiles and mycelium in one or more alternating layers.

8. The method of claim 1, wherein applying includes combining the substrate and mycelium.

9. The method of claim 1, wherein applying includes placing textiles and mycelium into a mold.

10. The method of claim 1, further comprising sterilizing the substrate prior to applying.

11. The method of claim 1, wherein the one or more textiles includes cotton.

12. The method of claim 1, wherein the one or more textiles includes polyester.

13. The method of claim 1, wherein the mycelium is of the genus Pleurotus or Ganoderma.

14. The method of claim 1, wherein allowing the mycelium to grow includes incubating the composite.

15. A mycelium-based biocomposite comprising:
a substrate including one or more textiles; and
mycelium,
wherein the mycelium is integrated into the substrate.

16. The biocomposite of claim 15, wherein the one or more textiles include one or more of cotton, polyester, nylon, and some combination thereof.

17. The biocomposite of claim 15, wherein the biocomposite is substantially fire resistant.

18. The biocomposite of claim 15, wherein the substrate has an average porosity of between 10-100 μm.

19. The biocomposite of claim 15, wherein the biocomposite has humidity absorption of between 2-10% at 65% relative humidity and 21° C.

20. The biocomposite of claim 15, wherein the biocomposite has humidity absorption of between 2-15% at 90% relative humidity and 21° C.
